(19) United States
(12) Patent Application Publication
(10) Pub. No.: US 2021/0353631 A1
(43) Pub. Date: Nov. 18, 2021

(54) 1,4-Benzoxazines for the Treatment of Cancers and Other Neurodegenerative Diseases

(71) Applicant: Neugeneron LLC, Dallas, TX (US)

(72) Inventor: Santosh D'Mello-Kamath, Dallas, TX (US)

(21) Appl. No.: 17/317,980

(22) Filed: May 12, 2021

Related U.S. Application Data
(60) Provisional application No. 63/023,544, filed on May 12, 2020.

(51) Int. Cl. A61K 31/538; A61K 45/06

(52) U.S. Cl. CPC A61K 31/538; A61K 45/06

(57) ABSTRACT

The present invention includes a method of treating a cancer or neurodegenerative disease in a subject in need thereof, the method comprising administering directly into the malignant glioma or intravenously to the subject a therapeutically effective amount of a compound of formula:

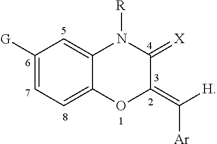

### CROSS-REFERENCE TO RELATED APPLICATIONS

This application claims priority to U.S. Provisional Application Ser. No. 63/023,544, filed May 12, 2020, the entire contents of which are incorporated herein by reference.

### TECHNICAL FIELD OF THE INVENTION

The present invention relates in general to the field of cancers, including brain cancers such as glioblastoma, and neurodegenerative diseases.

### STATEMENT OF FEDERALLY FUNDED RESEARCH

None.

### BACKGROUND OF THE INVENTION

Without limiting the scope of the invention, its background is described in connection with.

Neurodegenerative diseases, such as Alzheimer's disease (AD), Parkinson's disease (HD), and Huntington's disease (HD) are relatively prevalent and fatal brain diseases among older individuals which have a devastating impact on the patient, caregivers, and on society. Glioblastoma (GBM) is the most common primary brain tumor and is recognized as one of the most aggressive and lethal of human tumors. There are no effective treatments for either neurodegenerative diseases or GBM. While both disorders are thought to, in most cases, result from an interaction of genetic and environmental factors, and age is an important risk factor in both cases, the cellular outcome in neurodegenerative diseases and GBMs (or other types of cancers) is highly dissimilar. Specifically, neurodegenerative diseases are caused by the premature death of postmitotic neurons while GBM generally results from excessive cell survival. Consistent with the opposite effect on the cell, some clinical and epidemiological studies have described an inverse comorbidity of these two types of brain disorders^{1,2}. Not surprisingly, many genes that are upregulated in brain samples of patients with neurodegenerative diseases are downregulated in GBM and vice versa².

Despite recent advances, a need remains for novel methods of treating neurodegenerative diseases and/or GBM. What is needed are methods for the selective treatment of neurodegenerative diseases and GBM.

### SUMMARY OF THE INVENTION

In one embodiment, the present invention includes a method of treating a malignant glioma in a subject in need thereof, the method comprising administering directly into the malignant glioma or intravenously to the subject a therapeutically effective amount of a compound of formula:

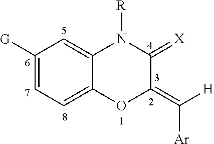

R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic; G is selected from H, C, N, halide, if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms; if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group. In one aspect, the effective amount of the compound is 1.0 μg to 1 g, about 1 mg to about 1000 mg, or from about 10 mg to about 100 mg, or from about 10 mg to about 50 mg, or from about 10 mg to about 25 mg of compound. In another aspect, further comprising adding one or more excipients selected from buffers, buffer salts, bulking agents, salts, surface active agents, acids, bases, saccharides, or binders. In another aspect, the compound is formulated into a composition comprising the compound at 0.1 wt % to about 20 wt %, from about 0.1 wt % to about 18 wt %, from about 0.1 wt % to about 16 wt %, from about 0.1 wt % to about 14 wt %, from about 0.1 wt % to about 12 wt %, from about 0.1 wt % to about 10 wt %, from about 0.1 wt % to about 8 wt %, from about 0.1 wt % to about 6 wt %, from about 0.1 wt % to about 4 wt %, from about 0.1 wt % to about 2 wt %, from about 0.1 wt % to about 1 wt %, from about 0.1 wt % to about 0.9 wt %, from about 0.1 wt % to about 0.8 wt %, from about 0.1 wt % to about 0.7 wt %, from about 0.1 wt % to about 0.6 wt %, from about 0.1 wt % to about 0.5 wt %, from about 0.1 wt % to about 0.4 wt %, from about 0.1 wt % to about 0.3 wt %, or from about 0.1 wt % to about 0.2 wt % of the total weight of the composition. In another aspect, the compound is at a level of about 0.1 wt %, about 0.2 wt %, about 0.3 wt %, about 0.4 wt %, about 0.5 wt %, about 0.6 wt %, about 0.7 wt %, about 0.8 wt %, or about 0.9 wt % based on the total weight of the composition. In another aspect, the malignant glioma is selected from the group consisting of glioblastoma, astrocytoma, oligodendroglioma, ependymoma, and juvenile pilocystic astrocytoma. In another aspect, the compound is adapted for intravenous, intranasal, intracranial, intrathecal, enteral, parenteral, or oral administration. In another aspect, the cancer is selected from at least one of a leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. In another aspect, the compound is administered as a single unit dose or in multiple doses over time. In another aspect, the method further comprises administering a therapy selected from the group consisting of immunotherapy, chemotherapy, radiotherapy, phototherapy, photodynamic therapy, surgery, nutritional therapy, ablative therapy, brachiotherapy, proton beam therapy, immunotherapy, cellular therapy and photon beam radiosurgical therapy. In another aspect, the compound is formulated into a nanoparticle, a nanovehicles, anexosome, liposome, or provided as a pro-drug. In another aspect, the administering comprises intravenous administration, and wherein the malignant glioma is a glioblastoma. In another aspect, the therapeutically effective amount of the compound is effective to reduce the activity of a DRAK1/STK17A serine-threonine kinase. In another aspect, the method further comprises administering a furan-thiazolidinedione, a pyridone-thiazolidinedione, or both.

In another embodiment, the present invention includes a method for treating a neurodegenerative diseases or degenerative neurological condition by providing a patient with an effective amount of an inhibitor of a DRAK1/STK17A or DRAK2/STK17B serine-threonine kinase. In one aspect, the inhibitor of a DRAK1/STK17A or DRAK2/STK17B serine-threonine kinase is a compound of formula:

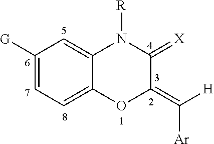

R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic; G is selected from H, C, N, halide, if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms; if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group. In one aspect, the neurodegenerative diseases or degenerative neurological conditions is a stroke or a traumatic brain injury. In another aspect, the method further comprises administering a furan-thiazolidinedione, a pyridone-thiazolidinedione, or both. In another aspect, the agent is biological, chemical, genetic, small RNAs, small DNAs, antisense oligonucleotides, or CRISPR.

In another embodiment, the present invention includes a method of preventing neuronal loss/neurodegeneration by providing an effective amount of an inhibitor of a DRAK1/STK17A serine-threonine kinase. In one aspect, the inhibitor of a DRAK1/STK17A or DRAK2/STK17B serine-threonine kinase is a compound of formula:

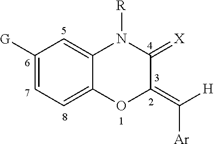

R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic; G is selected from H, C, N, halide, if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms; if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group. In another aspect, the method further comprises administering a furan-thiazolidinedione, a pyridone-thiazolidinedione, or both. In another aspect, the neurodegenerative diseases or degenerative neurological conditions is a stroke or a traumatic brain injury.

In another embodiment, the present invention includes an inhibitor of DRAK1/STK17A serine-threonine kinase, wherein the inhibitor is a 1,4-benzoxazine. In one aspect, the inhibitor is a compound of formula:

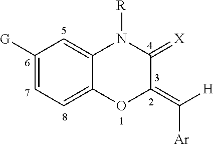

R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic; G is selected from H, C, N, halide, if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms; if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group. In one aspect, the inhibitor is (2Z)-6-amino-2-[(4-chlorophenyl)methylidene]-3,4-dihydro-2H-1,4-benzoxazin-3-one.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C, show: (FIG. 1A) Chemical structure of NGN-006. (FIG. 1B) Neuroprotection in cerebellar granule neurons (CGNs) induced to die by switching from high potassium (HK) to low potassium (LK) medium. HSB-13 and NGN-006 were added to LK-treated CGNs at 1, 5 and 25 uM concentrations. Viability was measured 24 h later and compared with control survival in HK). Results are means values from a single experiment performed in duplicate. Phase micrographs shows appearance of cultures with 25 uM drug at 24 h. (FIG. 1C) Neuroprotection in cortical neurons (CGNs) induced to die by treatment with homocysteic acid (HCA) which induces oxidative stress. Neurons were treated either with HCA alone or HCA with 25 uM HSB-13 or 5 uM and 25 uM NGN-006 for 18 h. Viability was measured 24 h later and compared with survival of control (untreated cells, Unt). Results are mean values from a single experiment performed in duplicate. Phase micrographs shows appearance with 25 uM drug at 24 h.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present invention uses 1,4-benzoxazine compounds using one specific example, a chemical compound, (2Z)-6-amino-2-[(4-chlorophenyl)methylidene]-3,4-dihydro-2H-1,4-benzoxazin-3-one, or NGN-006, which is part of a large class of related compounds, that can be used in the treatment of both neurodegenerative diseases and glioblastoma.

1,4 benzoxazine, also referred to herein as NGN-006, was synthesized by the present inventor as part of a drug discovery effort to identifying neuroprotective drugs. Chemically, NGN-006 is a 1,4 benzoxazine closely related to HSB-13, a compound the present inventor also described as having strong neuroprotective effects both in cell culture and in vivo models of neurodegenerative disease³. Like HSB-13, NGN-006 is shown herein as also having a neuroprotective effect. Surprisingly, given its structural similarity, NGN-006 does not inhibit any of the kinases that HSB-13 inhibits, which include CDKs, GSK3β and p38 MAPK and would, therefore, be detrimental of regular cellular activities, is instead a highly selective inhibitor of DRAK1/STK17A, a protein kinase that promotes apoptosis in some cell types. Recently, DRAK1/STK17A has emerged as a key player in the pathogenesis of GBM⁴⁻⁶. Thus, NGN-006, or derivatives are useful in the treatment of both neurodegenerative diseases and cancers, including brain cancers such as glioblastoma multiforme (GBM).

The present inventor recognized that recent studies have found that despite the obvious differences, neurodegenerative diseases and cancer share fundamental commonalities at the cellular and molecular level. For example, deregulated activation of cyclin-dependent kinases (CDKs), key promoters of cell proliferation, is a feature of many cancers, including GBM, but also occurs in experimental models of neurodegenerative disease and brains of patients with these diseases⁷. Chemical inhibitors of CDKs inhibit tumorigenesis in experimental models of cancer while also protecting neurons in models of neurodegenerative diseases^{7,8}. Protein misfolding and aggregation, a characteristic feature of many neurodegenerative diseases, is emerging as a key pathological feature in cancer. Best characterized in this regard is the p53 tumor suppressor gene, mutations of which contribute to GBM and a variety of other cancers⁹. Cancer-causing mutations of p53 result in its aggregation, an alteration that promotes tumorigenesis¹⁰⁻¹³. Interestingly, through direct interaction, aggregates of mutant p53 can induce aggregation of wild-type p53 and the closely-related p63 and p73 proteins in a prion-like fashion leading cancer experts to question whether cancer might be a prion disease¹⁴⁻¹⁶. It is well-established that development of neurodegenerative diseases, including AD, PD HD, and ALS, also involves a prion-like cell-to-cell transmission of specific misfolded proteins in the brain¹⁷⁻¹⁹. Interestingly, aggregation of β-amyloid protein (Aβ), a hallmark of AD neuropathology, is also a feature of some cancers where it is found along with TIAF1 (TGFB1-Induced Anti-Apoptotic Factor 1), a protein that has been implicated in the generation of amyloid plaques in AD²⁰⁻²² providing an example of “crosstalk” at the molecular level between AD and cancer. Endoplasmic reticulum (ER) stress/activation of the unfolded protein response (UPR) that characterizes a variety of neurodegenerative diseases has recently also been found to be a key feature of cancers²³⁻²⁵. Although yet to be identified, pharmacological agents to disaggregate the pathological aggregates or reduce ER stress/UPR activation have been considered as a therapeutic approach for neurodegenerative diseases as well as for cancers^{23,26}. Damage and impaired repair mechanisms of genomic and mitochondrial DNA are a key features in the pathogenesis of both neurodegenerative diseases and cancer²⁷⁻³¹. Oxidative stress and defective autophagy regulation are yet other commonalities between the two disorders^{32,33}. At the molecular level, the most compelling evidence of a genetic linkage between neurodegenerative disease and cancer comes from mutations in the AT-mutated (ATM) gene. ATM mutations cause Ataxia telangiectasia (AT), a disease displaying both neurodegeneration and cancer^{34,35}. Another convincing example is the PARK2 gene, which encodes parkin, an E3 ubiquitin ligase. While mutations of PARK2 are a common cause of autosomal recessive PD, there is widespread consensus that PARK2 is also a tumor suppressor, somatic mutations of which cause GBM and other cancers³⁶⁻⁴¹. It is noteworthy that ATM and PARK2 mutations that cause cancer often occur at the exact same residues that cause neurodegeneration^{36,42,43}. Deregulated expression or mutations in several other genes that play key roles in neurodegenerative disease, including α-synuclein, PTEN-induced kinase-1 (PINK1), leucine-rich repeat kinase-2 (LRRK2), microtubule-associated protein tau (MAPT), amyloid precursor protein (APP, from which Aβ is derived), presenilin 1/2 (PSEN1/2), hypoxia-inducting factor (HIF1), and cyclin-dependent kinase 5 (CDKS), have also been implicated in cancer⁴⁴⁻⁴⁸. Likewise, deregulation of genes involved in cancer, including p53, cyclin D, cyclin E, Pin1 and protein phosphatase 2a (PP2A) are similarly deregulated in neurodegeneration⁴⁴⁻⁴⁷. Taken together, these results suggest that there is overlap in the molecular and cellular mechanisms underlying the pathogenesis of neurodegenerative disease and cancer. Unlike the teachings hereinabove, the present invention is directed to inhibition of the DRAK1/STK17A serine/threonine kinase that promotes both neurodegeneration and tumorigenesis, specifically GBM.

DRAK1/STK17a: DRAK1/STK17a (human ortholog is STK17A) is a member of the recently described death-associated protein kinase (DAPK) family of serine-threonine kinases^{4,49,50}, a family comprised of five members, DAPK1, DAPK2, DAPK3, DRAK1, and DRAK2. Of these, DRAK1/STK17A and DRAK2/STK17B are more distantly related and less characterized members of the DAPK family. Although there are no publications on the expression pattern of DRAK1/STK17A in the normal brain, data from the Allen Brain Atlas and Cortecon websites indicate that it is expressed in the cortex and other brain regions. Recent studies have found that expression of DRAK1/STK17a is highly elevated in human GBM, as well as in other cancers^{51,52}. DRAK1/STK17a was found to be necessary for transformation in a well-established Drosophila model of GBM^{5,6}. Knockdown of DRAK1/STK17a or its inhibition by over-expression of a kinase-dead form suppressed the transformation of glial cells resulting from constitutive expression of epidermal growth factor receptor (EGFR) and PI-3 kinase⁶. Very interestingly, while essential for neoplastic proliferation of glial cells, knockdown of DRAK1/STK17a does not affect normal glial cell proliferation⁶. This suggests that, unlike many other anti-cancer drugs, DRAK1/STK17a inhibition may not affect normal cellular activity and functions and therefore be better tolerated by patients. DRAK1/STK17a acts downstream of EGFR activation in the neoplastic process leading to GBM^{4,5}. It may be noted that about 50% of GBMs are caused by EGFR gene amplification and increased EGFR expression. However, despite much effort to inhibit EGFR as a therapeutic strategy, these efforts have been unsuccessful⁵³⁻⁵⁵. Given this failure, inhibiting DRAK1, a target downstream of EGFR, might be an attractive alternative. Research in experimental models of GBM involving knockdown of DRAK1/STK17a support this concept^{5,6}.

Although its role in GBM pathogenesis is best documented, accumulating evidence indicates that DRAK1/STK17a promotes other types of cancers also. DRAK1/STK17a promotes head and neck cancers by disrupting TGF-β1-induced growth inhibition⁵¹. This involves interaction of elevated DRAK1/STK17a with SMAD3, which prevents SMAD3 from interacting with SMAD4, an interaction required for TGF-β1 signaling. Knockdown of DRAK1/STK17a permits TGF-β1 signaling and suppresses tumorigenicity in xenograft models in vivo⁵¹. DRAK1/STK17A also promotes cervical cancer⁵⁶. Low expression of DRAK1/STK17a has been found to correlate with high efficacy of radiotherapy against cervical cancer⁵⁶. Indeed, it has been suggested that DRAK1/STK17a represents a therapeutic biomarker for efficacy and prognosis in patients with cervical cancer treated with radiotherapy⁵⁶. Another study using ovarian cancer cells described that the efficacy of chemotherapy drugs was greatly increased by siRNA-mediated knockdown of DRAK1. Based on such findings, inhibition of DRAK1/STK17a could be an attractive therapeutic approach for cancers other than GBM^{4,5,51,52}.

Paradoxically, in view of its role in cancer where it promotes cell survival and growth, some studies have found that DRAK1/STK17a promotes apoptosis^{49,57,58}. Such pro-apoptotic activity provides a potential explanation for why its inhibition by NGN-006 protects neurons from death. Downregulation of DRAK1/STK17a upregulates the CDK inhibitory protein, p21^{WAF151}, a protein that studies from our lab^{59,60} and others⁶¹⁻⁶³ have shown to have strong neuroprotective effects. Although how DRAK1/STK17a can promote both apoptosis and tumorigenesis remains to be resolved, it is possible that this depends on differential posttranslational modifications, protein-protein interactions, and intracellular localization. For example, while its effect in promoting cancer appears to depend on cytoplasmic localization and through the regulation of the cytoskeleton, cytokinesis and cell adhesion, the pro-apoptotic action of DRAK1/STK17a appears to require nuclear localization^{4,57,58,64}. In the nucleus, DRAK1/STK17a interacts with p53 and stimulates p53 transcriptional activity in response to DNA-damage⁵⁷. One study described that DRAK1/STK17a translocates to the mitochondria where it interacts with the mitochondrial inner-membrane protein, adenine nucleotide translocase-2 (ANT-2), an anti-apoptotic oncoprotein⁵⁷. Inhibition of ANT-2 through such an interaction could explain the oncogenic action of DRAK1.

Based on the kinome-profiling analysis and emerging findings that strongly implicate DRAK1/STK17a as a key player in the development of GBM, NGN-006 is expected to kill and/or reduce proliferation of patient-derived GBM cells. The identification of DRAK1/STK17a as a therapeutic target for neurodegenerative disease is a novel aspect of the present invention that connects DRAK1/STK17a to neurodegenerative disease.

The data provided herewith shows results of using a chemical inhibitor with an unusually high level of selectivity for DRAK1. Even the closely-related DRAK2 protein or other members of the DAPK family, are not inhibited by NGN-006. DRAK1/STK17a has emerged as a novel therapeutic target for GMB, the most common adult brain cancer. NGN-006, or derivatives of it, represent a treatment for GBM by itself, but can also be used in combination with radiotherapy and/or other types of chemotherapeutic agents. Although most convincingly demonstrated for GBM, DRAK1/STK17a is involved in other types of cancers, therefore, NGN-006 has a broader utility in cancer treatment.

Based on the results herein, NGN-006 is expected to be protective in cell culture models of AD and HD. Thus, NGN-006 represents a starting point for effective treatments for AD and HD, and perhaps other neurodegenerative diseases, with compound of formula:

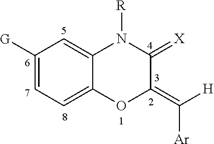

R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic;

G is selected from H, C, N, halide,

if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms;

if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and

Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group. Although shown to promote apoptosis in cycling cell types, the possibility that DRAK1/STK17a might be involved in neurodegenerative diseases is novel.

NGN-006 is neuroprotective. The present inventor, and others, discovered that certain 1,4 benzoxazine compounds were protective against neuronal loss in cell culture models of neurodegeneration. Among the neuroprotective 1,4 benzoxazine compounds identified, one compound, HSB-13 was selected, for further study. HSB-13 was not only protective in cell culture models, but also prevented neurodegeneration and improved behavioral performance in a mouse model of HD³. Additionally, HSB-13 was protective in a widely-used fly model of AD³. A patent covering composition of 1,4 benzoxazines and their use to protect against neurodegenerative diseases and conditions was granted in 2014 (U.S. Pat. No. 8,680,094; 1,4-benzoxazine compounds and derivatives thereof as therapeutic drugs for the treatment of neurodegenerative conditions, relevant portions incorporated herein by reference, including the synthetic methods for making HSB-13). Kinome profiling studies showed that the protein kinases most inhibited by HSB-13 were GSK3β, p38 MAPK, and cyclin-dependent kinases (CDKs).

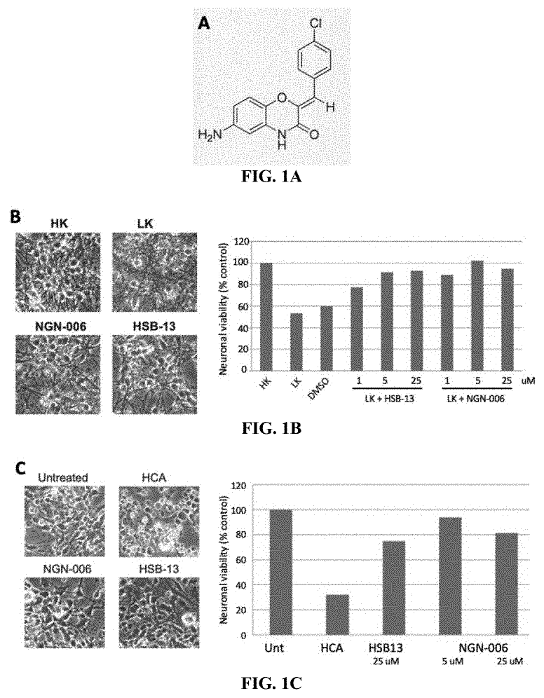

Based on the structure of HSB-13 addition compounds have been synthesized. Among these derivatives was NGN-006 (FIG. 1A). NGN-006 was tested in two cell culture models of neuronal death—(1) Switching of cerebellar granule neurons (CGNs) from medium containing depolarizing levels of potassium (high K+ or HK) to non-depolarizing (low K+ or LK) medium, and (2) cortical neurons treated with homocysteic acid (HCA) which induces oxidative stress, leading to cell death. These studies revealed that NGN-006 was protective in both models (FIGS. 1B and C). This was surprising because it was not expected that a highly selective kinase inhibitor could have the same overall effect as a broad inhibitor, HSB-13. FIGS. 1A to 1C, show: (FIG. 1A) Chemical structure of NGN-006. (FIG. 1B) Neuroprotection in cerebellar granule neurons (CGNs) induced to die by switching from high potassium (HK) to low potassium (LK) medium. HSB-13 and NGN-006 were added to LK-treated CGNs at 1, 5 and 25 uM concentrations. Viability was measured 24 h later and compared with control survival in HK). Results are means values from a single experiment performed in duplicate. Phase micrographs shows appearance of cultures with 25 uM drug at 24 h. (FIG. 1C) Neuroprotection in cortical neurons (CGNs) induced to die by treatment with homocysteic acid (HCA) which induces oxidative stress. Neurons were treated either with HCA alone or HCA with 25 uM HSB-13 or 5 uM and 25 uM NGN-006 for 18 h. Viability was measured 24 h later and compared with survival of control (untreated cells, Unt). Results are mean values from a single experiment performed in duplicate. Phase micrographs shows appearance with 25 uM drug at 24 h.

As shown in Table 1, NGN-006 is a highly-selective inhibitor of DRAK1. To obtain confirmation that NGN-006 acted like HSB-13, a kinome-profiling analyses was performed using the DiscoveRx/Eurofin platform. Surprisingly, NGN-006 did not inhibit any of the kinases that HSB-13 inhibited, but rather, inhibited DRAK1/STK17a (Table 1). Furthermore, of the 451 kinases against which it was tested, NGN-006 inhibited only DRAK1/STK17a substantially (defined as over 36% inhibition at 1 uM). Using the same criteria, GW5074, a 3′-substituted indolone commercially sold as a c-Raf inhibitor, that the inventor first^{66,68}, and subsequently others^{69,70}, described as neuroprotective, inhibited 21 of the 451 kinases when tested at the same time as NGN-006. Previous analyses of HSB-13 found that at 500 nM it inhibited 3 kinases out of a panel of 20 kinases it was tested against, whereas ASK-2A, another compound similar to HSB-13 and which is also neuroprotective, inhibited 5 of 20 kinases. All the kinases inhibited by HSB-13 were also inhibited by ASK-2A. Together, these results demonstrate for the first time that NGN-006 is a compound that acts differently from previously studied benzoxazines, and that it acts with an unusually high level of selectivity for DRAK1. It is notable that currently, and to the best of our knowledge, there are no commercially-available DRAK1/STK17a inhibitors.

TABLE 1
Kinome profiling results. NGN-006 was tested against a total
of 451 kinases. DRAK1/STK17a inhibition is shown.

| | Entrez | % |
|---|---|---|
| KINOMEscan gene symbol | gene eymbol | control |
| CIT | CIT | 100 |
| CLK1 | CLK1 | 79 |
| CLK2 | CLK2 | 100 |
| CLK3 | CLK3 | 92 |
| CLK4 | CLK4 | 100 |
| CSF1R | CSF1R | 100 |
| CSF1R -autoinhibited | CSF1R | 100 |
| CSK | CSK | 97 |
| CSNK1A1 | CSNK1A1 | 100 |
| CSNK1A1L | CSNK1A1L | 100 |
| CSNK1D | CSNKID | 100 |
| CSNK1E | CSNKIE | 89 |
| CSNK1G1 | CSNK1G1 | 100 |
| CSNK1G2 | CSNK1G2 | 100 |
| CSNK1G3 | CSNK1G3 | 84 |
| CSNK2A1 | CSNK2A1 | 100 |
| CSNK2A2 | CSNK2A2 | 100 |
| CTK | MATK | 100 |
| DAPK1 | DAPK1 | 100 |
| DAPK2 | DAPK2 | 86 |
| DAPK3 | DAPK3 | 88 |
| DCAMKL1 | DCLK1 | 100 |
| DCAMKL2 | DCLK2 | 98 |
| DCAMKL3 | DCLK3 | 100 |
| DDR1 | DDR1 | 100 |
| DDR2 | DDR2 | 100 |
| DLK | MAP3K12 | 100 |
| DMPK | DMPK | 100 |
| DMPK2 | CDC42BPG | 96 |
| DRAK1 | STK17A | 26 |
| DRAK2 | STK17B | 83 |
| DYRK1A | DYRK1A | 100 |
| DYRK1B | DYRK1B | 100 |
| DYRK2 | DYRK2 | 100 |
| EGFR | EGFR | 87 |
| EGFR(E746-A750del) | EGFR | 100 |
| EGFR(G719C) | EGFR | 100 |
| EGFR(G719S) | EGFR | 98 |
| EGFR(L747-E749del, A750P) | EGFR | 92 |
| EGFR(L747-S752del, P753S) | EGFR | 100 |
| EGFR(L747-T751del,Sins) | EGFR | 100 |

To test neuroprotection by NGN-006 in well-established cell culture models of AD and HD. NGN-006 was effective in LK-treated CGNs mimics the survival-promoting effect of neuronal activity during development. Elevated oxidative stress is a common feature of most, if not all neurodegenerative diseases.

NGN-006 was tested in two cell culture models of neuronal death that are disease-relevant utilizing primary cultures of cortical neurons: (1) Treatment with Aβ peptide, which is the most common cell culture model of AD. Briefly, after 5-6 days in vitro, primary cortical neuron cultures will be treated with 200 nM oligomeric Aβ₁₋₄₂. Scrambled or reversed at the same concentration will be used as control. Viability will be quantified by the Live/Dead assay, a widely-used assay and we have used in several studies previously. (2) Overexpression of polyQ-huntingtin (mut-Htt), which is the most common cell culture model of HD and one that we have used frequently^{59,60,72,73}. Briefly, after 4-5 days in culture, cortical neurons will be transfected with plasmids encoding GFP, wild-type Htt-GFP (Htt-Q₂₄) or mut-Htt-GFP (Htt-Q₁₃₈). The proportion of transfected cells GFP-positive) that are apoptotic (condensed or fragmented nuclei based on DAPI-staining) will be quantified. NGN-006 can be used at 5, 10 and 25 uM. Each experiment will be done in duplicate and three independent experiments will be performed. NGN-006 should be protective against Aβ and mut-Htt neurotoxicity. because in protection in HK/LK-CGN and HCA-cortical neuron models, these results are generally recapitulated in disease-relevant cell culture models.

To test the ability of NGN-006 to promote apoptosis in patient-derived primary glioblastoma cells. To assess the potential of NGN-006 as an anti-GBM drug using patient-derived GBM cells is preferred. A panel of 6-8 GBM patient-derived lines are treated with NGN-006 by itself or in combination with erlonib, an EGFR inhibitor to which they are generally resistant. NGN-006 can be used at concentrations of 5, 10, and 25 uM and whether it kills the cells by itself or sensitizes them to 1 uM erlotinib treatment will be assessed using AlamarBlue (ThermoFisher) or CellTiter—in duplicate and repeated three times. NGN-006 can also be tested on patient-derived GBM neurosphere lines (GBM9, GBM39, and SK297). These cells are all insensitive to erlotinib⁷⁴. Resistance to EGFR inhibition base been found to be mediated by c-Jun N-terminal kinase (JNK) activation. It is possible, but not a limitation of the present invention, that DRAK1/STK17a lies between EGFR and JNK activation in the tumorigenic signaling pathway leading to GBM. Although the relationship of its neoplastic effect to its apoptotic effect is unclear, the apoptotic activity of DRAK1/STK17a also requires JNK activation⁶⁴. In addition to viability assays, the effect of NGN-006 can be measured on GBM cell proliferation. This can be done using cell growth assays EdU incorporation (Life Technologies) and trypan blue-based cell counting.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Driver, J. A. Inverse association between cancer and neurodegenerative disease: review of the epidemiologic and biological evidence. Biogerontology 15, 547-557 (2014).
2. Ibáñez, K., Boullosa, C., Tabarés-Seisdedos, R., Baudot, A. & Valencia, A. Molecular evidence for the inverse comorbidity between central nervous system disorders and cancers detected by transcriptomic meta-analyses. PLoS Genet. 10, e1004173 (2014).
3. Wang, L. et al. Identification of novel 1,4-benzoxazine compounds that are protective in tissue culture and in vivo models of neurodegeneration. J. Neurosci. Res. 88, 1970-1984 (2010).
4. Lathia, J. D. Drak, Drak, Goose: A New Signaling Axis in Glioblastoma. Cancer Res. 79, 1036-1037 (2019).
5. Read, R. D., Cavenee, W. K., Furnari, F. B. & Thomas, J. B. A drosophila model for EGFR-Ras and PI3K-dependent human glioma. PLoS Genet. 5, e1000374 (2009).
6. Chen, A. S. et al. Drak/STK17A Drives Neoplastic Glial Proliferation through Modulation of MRLC Signaling. Cancer Res. 79, 1085-1097 (2019).
7. Malumbres, M., Pevarello, P., Barbacid, M. & Bischoff, J. R. CDK inhibitors in cancer therapy: what is next? Trends in Pharmacological Sciences 29, 16-21 (2008).
8. Monaco, E. A. & Vallano, M. L. Cyclin-dependent kinase inhibitors: cancer killers to neuronal guardians. Curr. Med. Chem. 10, 367-379 (2003).
9. Zhang, Y. et al. The p53 Pathway in Glioblastoma. Cancers (Basel) 10, (2018).
10. Gong, H. et al. Amyloidogenicity of p53: a hidden link between protein misfolding and cancer. Curr. Protein Pept. Sci. 16, 135-146 (2015).
11. de Oliveira, G. A. P., Rangel, L. P., Costa, D. C. & Silva, J. L. Misfolding, Aggregation, and Disordered Segments in c-Abl and p53 in Human Cancer. Front Oncol 5, 97 (2015).
12. Kanapathipillai, M. Treating p53 Mutant Aggregation-Associated Cancer. Cancers (Basel) 10, (2018).
13. Cordeiro, Y., Vieira, T., Kovachev, P. S., Sanyal, S. & Silva, J. L. Modulation of p53 and prion protein aggregation by RNA. Biochim Biophys Acta Proteins Proteom 1867, 933-940 (2019).
14. Rangel, L. P., Costa, D. C. F., Vieira, T. C. R. G. & Silva, J. L. The aggregation of mutant p53 produces prion-like properties in cancer. Prion 8, 75-84 (2014).
15. Costa, D. C. F. et al. Aggregation and Prion-Like Properties of Misfolded Tumor Suppressors: Is Cancer a Prion Disease? Cold Spring Harb Perspect Biol 8, (2016).
16. Silva, J. L., De Moura Gallo, C. V., Costa, D. C. F. & Rangel, L. P. Prion-like aggregation of mutant p53 in cancer. Trends Biochem. Sci. 39, 260-267 (2014).
17. Kretzschmar, H. & Tatzelt, J. Prion disease: a tale of folds and strains. Brain Pathol. 23, 321-332 (2013).
18. Grad, L. I., Fernando, S. M. & Cashman, N. R. From molecule to molecule and cell to cell: prion-like mechanisms in amyotrophic lateral sclerosis. Neurobiol. Dis. 77, 257-265 (2015).
19. Wemheuer, W. M., Wrede, A. & Schulz-Schaeffer, W. J. Types and Strains:

Their Essential Role in Understanding Protein Aggregation in Neurodegenerative Diseases. Front Aging Neurosci 9, 187 (2017).
20. Chang, J.-Y. et al. TIAF1 self-aggregation in peritumor capsule formation, spontaneous activation of SMAD-responsive promoter in p53-deficient environment, and cell death. Cell Death Dis 3, e302 (2012).
21. Hong, Q. et al. Self-aggregating TIAF1 in lung cancer progression. Transl Respir Med 1, 5 (2013).
22. Lee, M.-H. et al. TGF-β induces TIAF1 self-aggregation via type II receptor-independent signaling that leads to generation of amyloid β plaques in Alzheimer's disease. Cell Death Dis 1, e110 (2010).
23. da Silva, D. C., Valentão, P., Andrade, P. B. & Pereira, D. M. Endoplasmic reticulum stress signaling in cancer and neurodegenerative disorders: Tools and strategies to understand its complexity. Pharmacol. Res. 155, 104702 (2020).
24. Peñaranda Fajardo, N. M., Meijer, C. & Kruyt, F. A. E. The endoplasmic reticulum stress/unfolded protein response in gliomagenesis, tumor progression and as a therapeutic target in glioblastoma. Biochem. Pharmacol. 118, 1-8 (2016).
25. Hetz, C. & Saxena, S. ER stress and the unfolded protein response in neurodegeneration. Nat Rev Neurol 13, 477-491 (2017).
26. Oakes, S. A. Endoplasmic Reticulum Stress Signaling in Cancer Cells. Am. J. Pathol. (2020) doi:10.1016/j.ajpath.2020.01.010.
27. Thadathil, N., Hori, R., Xiao, J. & Khan, M. M. DNA double-strand breaks: a potential therapeutic target for neurodegenerative diseases. Chromosome Res. (2019) doi:10.1007/s10577-019-09617-x.
28. Maiuri, T. et al. DNA Damage Repair in Huntington's Disease and Other Neurodegenerative Diseases. Neurotherapeutics (2019) doi:10.1007/s13311-019-00768-7.
29. Zhu, Z. & Wang, X. Significance of Mitochondria DNA Mutations in Diseases. Adv. Exp. Med. Biol. 1038, 219-230 (2017).
30. Moro, L. Mitochondrial Dysfunction in Aging and Cancer. J Clin Med 8, (2019).
31. Kiwerska, K. & Szyfter, K. DNA repair in cancer initiation, progression, and therapy—a double-edged sword. J. Appl. Genet. 60, 329-334 (2019).
32. Gomez, M. & Germain, D. Cross talk between SOD1 and the mitochondrial UPR in cancer and neurodegeneration. Mol. Cell. Neurosci. 98, 12-18 (2019).
33. Zhang, W. et al. The emerging role of oxidative stress in regulating autophagy: applications of cancer therapy. Cell. Mol. Biol. (Noisy-le-grand) 63, 67-76 (2017).
34. Staropoli, J. F. Tumorigenesis and neurodegeneration: two sides of the same coin? Bioessays 30, 719-727 (2008).
35. Lavin, M. F. Ataxia-telangiectasia: from a rare disorder to a paradigm for cell signalling and cancer. Nat. Rev. Mol. Cell Biol. 9, 759-769 (2008).
36. Veeriah, S. et al. Somatic mutations of the Parkinson's disease-associated gene PARK2 in glioblastoma and other human malignancies. Nat. Genet. 42, 77-82 (2010).
37. Denison, S. R. et al. Alterations in the common fragile site gene Parkin in ovarian and other cancers. Oncogene 22, 8370-8378 (2003).
38. Wang, F. et al. Parkin gene alterations in hepatocellular carcinoma. Genes Chromosomes Cancer 40, 85-96 (2004).
39. Wang, F. et al. Parkin gene alterations in hepatocellular carcinoma. Genes Chromosomes Cancer 40, 85-96 (2004).
40. Cesari, R. et al. Parkin, a gene implicated in autosomal recessive juvenile parkinsonism, is a candidate tumor suppressor gene on chromosome 6q25-q27. Proc. Natl. Acad. Sci. U.S.A. 100, 5956-5961 (2003).
41. Picchio, M. C. et al. Alterations of the tumor suppressor gene Parkin in non-small cell lung cancer. Clin. Cancer Res. 10, 2720-2724 (2004).
42. Shiloh, Y. & Rotman, G. Ataxia-telangiectasia and the ATM gene: linking neurodegeneration, immunodeficiency, and cancer to cell cycle checkpoints. J. Clin. Immunol. 16, 254-260 (1996).
43. Veeriah, S., Morris, L., Solit, D. & Chan, T. A. The familial Parkinson disease gene PARK2 is a multisite tumor suppressor on chromosome 6q25.2-27 that regulates cyclin E. Cell Cycle 9, 1451-1452 (2010).
44. Houck, A. L., Seddighi, S. & Driver, J. A. At the Crossroads Between Neurodegeneration and Cancer: A Review of Overlapping Biology and Its Implications. Curr Aging Sci 11, 77-89 (2018).
45. Gargini, R., Segura-Collar, B. & Sánchez-Gómez, P. Novel Functions of the Neurodegenerative-Related Gene Tau in Cancer. Front Aging Neurosci 11, 231 (2019).
46. Seo, J. & Park, M. Molecular crosstalk between cancer and neurodegenerative diseases. Cell. Mol. Life Sci. (2019) doi:10.1007/s00018-019-03428-3.
47. Plun-Favreau, H., Lewis, P. A., Hardy, J., Martins, L. M. & Wood, N. W. Cancer and neurodegeneration: between the devil and the deep blue sea. PLoS Genet. 6, e1001257 (2010).
48. Galvão, F., Grokoski, K. C., da Silva, B. B., Lamers, M. L. & Siqueira, I. R. The amyloid precursor protein (APP) processing as a biological link between Alzheimer's disease and cancer. Ageing Res. Rev. 49, 83-91 (2019).
49. Inbal, B., Shani, G., Cohen, O., Kissil, J. L. & Kimchi, A. Death-associated protein kinase-related protein 1, a novel serine/threonine kinase involved in apoptosis. Mol.

Cell. Biol. 20, 1044-1054 (2000).
50. Sanjo, H., Kawai, T. & Akira, S. DRAKs, novel serine/threonine kinases related to death-associated protein kinase that trigger apoptosis. J. Biol. Chem. 273, 29066-29071 (1998).
51. Park, Y. et al. Cytoplasmic DRAK1 overexpressed in head and neck cancers inhibits TGF-β1 tumor suppressor activity by binding to Smad3 to interrupt its complex formation with Smad4. Oncogene 34, 5037-5045 (2015).
52. Mao, P. et al. Serine/threonine kinase 17A is a novel candidate for therapeutic targeting in glioblastoma. PLoS ONE 8, e81803 (2013).
53. Lo, H.-W. EGFR-targeted therapy in malignant glioma: novel aspects and mechanisms of drug resistance. Curr Mol Pharmacol 3, 37-52 (2010).
54. Taylor, T. E., Furnari, F. B. & Cavenee, W. K. Targeting EGFR for treatment of glioblastoma: molecular basis to overcome resistance. Curr Cancer Drug Targets 12, 197-209 (2012).
55. Gadji, M. et al. EGF receptor inhibitors in the treatment of glioblastoma multiform: old clinical allies and newly emerging therapeutic concepts. Eur. J. Pharmacol. 625, 23-30 (2009).
56. Wei, W. & Liu, C. Prognostic and predictive roles of microRNA-411 and its target STK17A in evaluating radiotherapy efficacy and their effects on cell migration and invasion via the p53 signaling pathway in cervical cancer. Mol Med Rep 21, 267-281 (2020).
57. Oue, Y., Murakami, S., Isshiki, K., Tsuji, A. & Yuasa, K. Intracellular localization and binding partners of death associated protein kinase-related apoptosis-inducing protein kinase 1. Biochem. Biophys. Res. Commun. 496, 1222-1228 (2018).
58. Kojima, H. et al. rDRAK1, a Novel Kinase Related to Apoptosis, Is Strongly Expressed in Active Osteoclasts and Induces Apoptosis. J. Biol. Chem. 276, 19238-19243 (2001).
59. Mallick, S. & D'Mello, S. R. JAZ (Znf346), a SIRT1-interacting protein, protects neurons by stimulating p21 (WAF/CIP1) protein expression. J. Biol. Chem. 289, 35409-35420 (2014).
60. Louis Sam Titus, A. S. C. et al. Reduced Expression of Foxpl as a Contributing Factor in Huntington's Disease. J. Neurosci. 37, 6575-6587 (2017).
61. Brochier, C. et al. Specific acetylation of p53 by HDAC inhibition prevents DNA damage-induced apoptosis in neurons. J. Neurosci. 33, 8621-8632 (2013).
62. Sleiman, S. F. et al. Histone Deacetylase Inhibitors and Mithramycin A Impact a Similar Neuroprotective Pathway at a Crossroad between Cancer and Neurodegeneration. Pharmaceuticals (Basel) 4, 1183-1195 (2011).
63. Sleiman, S. F. et al. Mithramycin is a gene-selective Sp1 inhibitor that identifies a biological intersection between cancer and neurodegeneration. J. Neurosci. 31, 6858-6870 (2011).
64. Manivannan, P., Reddy, V., Mukherjee, S., Clark, K. N. & Malathi, K. RNase L Induces Expression of A Novel Serine/Threonine Protein Kinase, DRAK1, to Promote Apoptosis. Int J Mol Sci 20, (2019).
65. Harper, S. J. & Wilkie, N. MAPKs: new targets for neurodegeneration. Expert Opin. Ther. Targets 7, 187-200 (2003).
66. Chin, P. C. & D'Mello, S. R. Brain chemotherapy from the bench to the clinic: targeting neuronal survival with small molecule inhibitors of apoptosis. Front. Biosci. 10, 552-568 (2005).
67. D'Mello, S. R. & Chin, P. C. Treating neurodegenerative conditions through the understanding of neuronal apoptosis. Curr Drug Targets CNS Neurol Disord 4, 3-23 (2005).
68. Chin, P. C. et al. The c-Raf inhibitor GW5074 provides neuroprotection in vitro and in an animal model of neurodegeneration through a MEK-ERK and Akt-independent mechanism. J. Neurochem. 90, 595-608 (2004).
69. Li, J. et al. The Raf-1 inhibitor GW5074 and the ERK1/2 pathway inhibitor U0126 ameliorate PC12 cells apoptosis induced by 6-hydroxydopamine. Pharmazie 67, 718-724 (2012).
70. Liu, Z. et al. Inhibitors of LRRK2 kinase attenuate neurodegeneration and Parkinson-like phenotypes in Caenorhabditis elegans and Drosophila Parkinson's disease models. Hum. Mol. Genet. 20, 3933-3942 (2011).
71. Dastidar, S. G. et al. Isoform-specific toxicity of Mecp2 in postmitotic neurons: suppression of neurotoxicity by FoxG1. J. Neurosci. 32, 2846-2855 (2012).
72. Bardai, F. H. et al. Disassociation of histone deacetylase-3 from normal huntingtin underlies mutant huntingtin neurotoxicity. J. Neurosci. 33, 11833-11838 (2013).
73. Verma, P., Pfister, J. A., Mallick, S. & D'Mello, S. R. HSF1 protects neurons through a novel trimerization- and HSP-independent mechanism. J. Neurosci. 34, 1599-1612 (2014).
74. Guo, G. et al. A TNF-JNK-Axl-ERK signaling axis mediates primary resistance to EGFR inhibition in glioblastoma. Nat. Neurosci. 20, 1074-1084 (2017).

What is claimed is:

1. A method of treating a malignant glioma in a subject in need thereof, the method comprising administering directly into the malignant glioma or intravenously to the subject a therapeutically effective amount of a compound of formula: 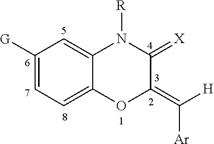
R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic;
G is selected from H, C, N, halide,
if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms;
if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and
Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group.

2. The method of claim 1, wherein the effective amount of the compound is 1.0 μg to 1 g, about 1 mg to about 1000 mg, or from about 10 mg to about 100 mg, or from about 10 mg to about 50 mg, or from about 10 mg to about 25 mg of compound.

3. The method of claim 1, further comprising adding one or more excipients selected from buffers, buffer salts, bulking agents, salts, surface active agents, acids, bases, saccharides, or binders.

4. The method of claim 1, wherein the compound is formulated into a composition comprising the compound at 0.1 wt % to about 20 wt %, from about 0.1 wt % to about 18 wt %, from about 0.1 wt % to about 16 wt %, from about 0.1 wt % to about 14 wt %, from about 0.1 wt % to about 12 wt %, from about 0.1 wt % to about 10 wt %, from about 0.1 wt % to about 8 wt %, from about 0.1 wt % to about 6 wt %, from about 0.1 wt % to about 4 wt %, from about 0.1 wt % to about 2 wt %, from about 0.1 wt % to about 1 wt %, from about 0.1 wt % to about 0.9 wt %, from about 0.1 wt % to about 0.8 wt %, from about 0.1 wt % to about 0.7 wt %, from about 0.1 wt % to about 0.6 wt %, from about 0.1 wt % to about 0.5 wt %, from about 0.1 wt % to about 0.4 wt %, from about 0.1 wt % to about 0.3 wt %, or from about 0.1 wt % to about 0.2 wt % of the total weight of the composition.

5. The method of claim 1, wherein the compound is at a level of about 0.1 wt %, about 0.2 wt %, about 0.3 wt %, about 0.4 wt %, about 0.5 wt %, about 0.6 wt %, about 0.7 wt %, about 0.8 wt %, or about 0.9 wt % based on the total weight of the composition.

6. The method of claim 1, wherein the malignant glioma is selected from the group consisting of glioblastoma, astrocytoma, oligodendroglioma, ependymoma, and juvenile pilocystic astrocytoma.

7. The method of claim 1, wherein the compound is adapted for intravenous, intranasal, intracranial, intrathecal, enteral, parenteral, or oral administration.

8. The method of claim 1, wherein the cancer is selected from at least one of a leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.

9. The method of claim 1, wherein the compound is administered as a single unit dose or in multiple doses over time.

10. The method of claim 1, further comprising administering a therapy selected from the group consisting of immunotherapy, chemotherapy, radiotherapy, phototherapy, photodynamic therapy, surgery, nutritional therapy, ablative therapy, brachiotherapy, proton beam therapy, immunotherapy, cellular therapy and photon beam radiosurgical therapy.

11. The method of claim 14, wherein the compound is formulated into a nanoparticle, a nanovehicles, anexosome, liposome, or provided as a pro-drug.

12. The method of claim 1, wherein administering comprises intravenous administration, and wherein the malignant glioma is a glioblastoma.

13. The method of claim 16, wherein the therapeutically effective amount of the compound is effective to reduce the activity of a DRAK1/STK17A serine-threonine kinase.

14. The method of claim 1, further comprising administering a furan-thiazolidinedione, a pyridone-thiazolidinedione, or both.

15. A method for treating a neurodegenerative diseases or degenerative neurological condition by providing a patient with an effective amount of an inhibitor of a DRAK1/STK17A or DRAK2/STK17B serine-threonine kinase.

16. The method of claim 15, wherein the inhibitor of a DRAK1/STK17A or DRAK2/STK17B serine-threonine kinase is a compound of formula: 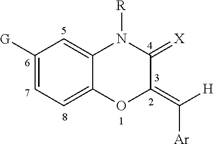
R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic;
G is selected from H, C, N, halide,
if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms;
if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and
Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group.

17. The method of claim 15, wherein the neurodegenerative diseases or degenerative neurological conditions is a stroke or a traumatic brain injury.

18. The method of claim 15, further comprising administering a furan-thiazolidinedione, a pyridone-thiazolidinedione, or both.

19. The method of claim 15, wherein the agent is biological, chemical, genetic, small RNAs, small DNAs, antisense oligonucleotides, or CRISPR.

20. A method of preventing neuronal loss/neurodegeneration by providing an effective amount of an inhibitor of a DRAK1/STK17A serine-threonine kinase.

21. The method of claim 20, wherein the inhibitor of a DRAK1/STK17A or DRAK2/STK17B serine-threonine kinase is a compound of formula: 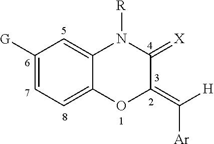
R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic;
G is selected from H, C, N, halide,
if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms;
if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and
Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group.

22. The method of claim 20, further comprising administering a furan-thiazolidinedione, a pyridone-thiazolidinedione, or both.

23. The method of claim 20, wherein the neurodegenerative diseases or degenerative neurological conditions is a stroke or a traumatic brain injury.

24. An inhibitor of DRAK1/STK17A serine-threonine kinase, wherein the inhibitor is a 1,4-benzoxazine.

25. The inhibitor of claim 24, wherein the inhibitor is a compound of formula: 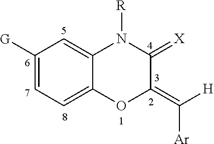
R is selected from the group consisting of: hydrogen, C1-C6 alkyl, alkenes or alkynes straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic;
G is selected from H, C, N, halide,
if G is N, then further comprising R2 and R3 each independently selected from the group consisting of: hydrogen; C1-C6 alkyl straight chained, branched or cyclic; substituted C1-C6 alkyl straight chained, branched or cyclic; C1-C6 alkenyl straight chained, branched or cyclic; or substituted C1-C6 alkenyl straight chained, branched or cyclic, R2 and R3 taken together may form a ring containing from 3 to 6 carbon atoms and optionally one or more heteroatoms;
if G is C, then further comprising R4 and R5 and R6 each independently selected from the group consisting of: hydrogen; C1-C6 alkyl, alkenes or alkynes or straight chained, branched or cyclic; substituted C1-C6 alkyl, alkenes or alkynes straight chained, branched or cyclic; C1-C6 alkenyl straight chained, branched or cyclic; or substituted C1-C6 alkenyl straight chained, branched or cyclic, R4 and R5 and R6 taken together may form a ring containing from 3 to 6 carbon atoms and optionally one or more heteroatoms; and
Ar is selected from the group consisting of phenyl, substituted phenyl, naphthyl, substituted naphthyl, biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, heteroaryl, substituted heteroaryl, 3,5-dibromophenyl, 3,5-dibromo-4-hydroxyphenyl, 3,5-dibromo-4-acetoxyphenyl, 3,4,5-trimethoxyphenyl, 4-dimethylaminophenyl, 2,5-dimethoxyphenyl, thiophen-3-yl, thiophen-2-yl, pyrrol-2-yl, pyridin-2-yl; and 3-indolyl; X is O or S; R is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, and an oxo group; G is selected from the group consisting of H, a C1-C6 Alkyl group, a C1-C6 Alkenyl group, a halo group, a substituted C1-C6 alkyl group, a substituted C1-C6 alkenyl group, a carbonyl group, a carbonate ester group, an C1-C6 ether group, an C1-C6 ester group, an C1-C6 alkyl alkanoate group, an C1-C6 alkoxy group, a keto group, an oxo group, a NO₂ containing group, and an amine containing group.

26. The inhibitor of claim 24, wherein the inhibitor is (2Z)-6-amino-2-[(4-chlorophenyl)methylidene]-3,4-dihydro-2H-1,4-benzoxazin-3-one.
